(19) United States
(12) Patent Application Publication
(10) Pub. No.: US 2002/0110905 A1
(43) Pub. Date: Aug. 15, 2002

(54) Perfusion system for cultured cells

(76) Inventors: Emilio Barbera-Guillem, Powell, OH (US); M. Bud Nelson, Worthington, OH (US)

Correspondence Address: ADMINISTRATOR, BioCrystal Ltd., 575 McCorkle Boulevard, Westerville, OH, 43082-8888, US

(21) Appl. No.: 09/784,533

(22) Filed: Feb. 15, 2001

(51) Int. Cl. C12M 3/00

(52) U.S. Cl. 435/294.1; 435/286.5; 435/297.2

(57) ABSTRACT

An in vitro system for perfusion of cultured cells comprising one or more cell culture devices, one or more reservoirs, and a perfusion mechanism for providing a fluid flow between the one or more cell culture devices and the one or more reservoirs. Also provided is a method of using the in vitro system to contact cultured cells, contained in the one or more cell culture devices, with a biological substance. The biological substance may be mixed with medium, and then the medium may be circulated through the one or more cell culture devices so that the biological substance contacts cultured cells contained in the one or more cell culture devices.

### FIELD OF THE INVENTION

The present invention generally relates to the field of cell culture; and more particularly to a perfusion system for cultured cells, for treating cultured cells with one or more biological substances, and for collecting one or more products secreted by cultured cells.

### Background of the Invention

Genomics, proteomics, drug discovery, the health aids industry, and pharmaceutics are generating a need for expanded versatility in developing and testing biological substances (e.g., including, but not limited to, genetic vectors, vaccines, genetic sequences, drugs, therapeutic agents, cosmetics, growth factors, cytokines, immunotoxins, recombinant products, chemicals, enzymes, monoclonal antibodies, cell modulating agents, viruses, reagents, nutraceuticals, and the like). One reason given for continued reliance on animal models for testing biological substances is the lack of relevant in vitro models that are able to mimic in vivo physiology (e.g., circulation and respiration). For example, testing a biological substance in a static cell culture fails to model the concentration effects and fluid flow effects (e.g., time of exposure) resulting from the process of circulation of a biological substance in body fluids encountered in vivo. Further, a static cell culture fails to model the in vivo metabolism of a biological substance as it is periodically circulated and contacts cells of a specific cell type, as well as a heterogeneous cell population of multiple cell types (e.g., as may be found in a tissue or organ). It may often be desirable to evaluate the response (pharmacological and/or biological) of eukaryotic cells after treatment with a biological substance; and additionally to evaluate the responses in a multitude of eukaryotic cells being treated simultaneously (e.g., in parallel). However, such responses in vivo are dependent upon the changing concentration of, and time of exposure to, a biological substance; and hence, cannot be accurately modeled in a static cell culture.

It is apparent to those skilled in the art that eukaryotic cells are capable of producing secreted products of commercial value. For example, monoclonal antibodies may be produced by eukaryotic hybridoma cells cultured in vitro. Additionally, since recombinant techniques have become routine, it is common for gene products to be expressed in cultured eukaryotic cells in vitro, and then secreted into the cell culture medium. However, conventional methods for harvesting such secreted products from cell cultures typically require that the cell cultures be disrupted (e.g., centrifugation of the cell cultures to achieve a separation between cultured cells and cell culture medium). Further, this “harvesting” phase represents additional time in which the cells are removed from a controlled environment, and hence, represents additional time during which a cell culture is unable able to mimic in vivo physiology (e.g., circulation and respiration).

A further limitation of conventional cell culture devices (e.g., tissue culture flasks), due to the relative inefficient gas transfer through the screw cap, is the requirement of a large volume of air space (relative to the growth surface; hence, the overall size of a tissue culture flask is rather bulky), and the dependency on a supply of gases (one or more of O₂, CO₂, and the like) that are pumped into the controlled environment (e.g., tissue culture incubator) in which the conventional cell culture devices are incubated. A means for more efficient gas transfer for cultured cells than provided by conventional cell culture devices (e.g., tissue culture flask or petri dish as provided by the header space of the device) would more accurately mimic respiration of cells in vivo.

Thus, there is a need for an in vitro system for perfusion of cultured cells in which the cultured cells may be exposed to a change in concentration of one or more biological substances, or a change in exposure to one or more biological substances, and a combination thereof. Additionally, there is a need for an in vitro system for perfusion of cultured cells that obviates the requirement for a supply of gases to be pumped into the environment of the cultured cells.

### SUMMARY OF THE INVENTION

It is a primary object to provide an in vitro system for perfusion of cultured cells with one or more biological substances added to the system.

It is another object to provide an in vitro system for perfusion of cultured cells that provides for circulation of medium with respect to the cultured cells, and further provides for respiration through a gas-permeable membrane in providing a more efficient transfer of gases to cultured cells than the transfer of gases achieved through a header space above cells as found in a conventional cell culture device.

It is another object of the present invention to provide an in vitro system for perfusion of cultured cells that obviates the requirement for a supply of gases to be pumped into the environment of the cultured cells.

It is another object of the present invention to provide an in vitro system for perfusion of cultured cells with one or more biological substances added to the system, wherein the system comprises a single cell culture device.

It is another object of the present invention to provide an in vitro system for perfusion of cultured cells with one or more biological substances added to the system, wherein the system comprises a plurality of cell culture devices.

It is another object of the present invention to provide an in vitro system for perfusion of cultured cells with one or more biological substances added to the system, wherein the system comprises cultured cells of a single cell type.

It is another object of the present invention to provide an in vitro system for perfusion of cultured cells with one or more biological substances added to the system, wherein the system comprises a plurality of cell types which comprise the cultured cells.

It is another object of the present invention to provide an in vitro system for perfusion of cultured cells so that one or more products secreted from the cultured cells may be evaluated and/or harvested without the need to disrupt a cell culture.

Briefly, the in vitro system for perfusion of cultured cells according to the present invention comprises: one or more cell culture devices containing cultured cells in a medium (preferably comprising a cell culture medium, but may include a physiologically acceptable solution other than cell culture medium known in the art for contacting cultured cells), wherein each cell culture device has an inlet port and an outlet port, and at least one gas permeable membrane; one or more reservoirs; a perfusion mechanism for providing a fluid flow communication between the one or more cell culture devices and the one or more reservoirs, and for circulating the medium by flowing the medium through an inlet port into each cell culture device in the system so that medium accesses the chamber of the cell culture device in contacting the cultured cells, and flowing medium out of the chamber of the cell culture device via an outlet port. The in vitro system for perfusion of cultured cells according to the present invention may further comprise a component selected from the group consisting of a rack for accommodating the one or more cell culture devices, a manifold for regulating the flow rate of medium into an inlet port, a manifold for regulating the flow rate of medium out of an outlet port, a sampling port by which a sample of medium being circulated through the perfusion mechanism may be withdrawn from the fluid flow of the in vitro system, a harvesting mechanism in operative communication with the fluid flow (e.g., for harvesting a secreted product from the medium flowed out of the one or more cell culture devices), one or more in-line sensors in operative communication with the fluid flow, a housing, a microprocessor for controlling functions and programmable operations of the in vitro system for perfusion of cultured cells, and a combination thereof.

These and other objects and advantages of the invention will become more apparent from the following detailed description taken in conjunction with the accompanying drawings.

### BRIEF DESCRIPTION OF THE DRAWINGS

FIG. 1 is a block diagram of an embodiment of an in vitro system for perfusion of cultured cells according to the present invention.

FIG. 2 is a block diagram of an embodiment of an in vitro system for perfusion of cultured cells according to the present invention.

FIG. 3 is an exploded view of the block diagrams illustrated in FIGS. 1 and 2, showing an embodiment of an in vitro system for perfusion of cultured cells according to the present invention.

FIG. 4 is an exploded view of the block diagrams illustrated in FIGS. 1 and 2, showing an embodiment of an in vitro system for perfusion of cultured cells according to the present invention.

FIG. 5 is a schematic illustration of an embodiment of the cell culture device of the in vitro system for perfusion of cultured cells according to the present invention.

FIG. 6 is a schematic illustration of an embodiment of the in vitro system for perfusion of cultured cells according to the present invention.

FIG. 7 is a schematic illustration of an embodiment of the in vitro system for perfusion of cultured cells according to the present invention.

FIG. 8 is a schematic illustration of an embodiment of the in vitro system for perfusion of cultured cells according to the present invention.

FIG. 9 is a schematic illustration of an embodiment of the in vitro system for perfusion of cultured cells according to the present invention.

FIG. 10 is a block diagram of a control system for the in vitro system for perfusion of cultured cells according to the present invention.

### DETAILED DESCRIPTION OF THE INVENTION

### Definitions

The term “tissue culture medium” is used herein, for the purposes of the specification and claims, to mean a liquid solution which is used to provide sufficient nutrients (e.g., vitamins, amino acids, essential nutrients, salts, and the like) and properties (e.g., osmolarity, buffering) to maintain living cells (preferably, eukaryotic cells) and support their growth. Various formulations of commercially available tissue culture medium are known to those skilled in the art. The term “cell culture medium” is used herein, for the purposes of the specification and claims, to mean tissue culture medium that has been incubated or contacted with cultured cells; and more preferably refers to tissue culture medium that further comprises substances secreted or excreted by cultured cells as a result of culturing the cells in the presence of the tissue culture medium. “Medium” is used herein, for the purposes of the specification and claims, to mean a fluid comprising tissue culture medium, cell culture medium, a physiologically acceptable solution, or a combination thereof. The medium may further comprise a biological substance, a secreted product, or a combination thereof. “A physiologically acceptable solution” comprises a fluid, other than tissue culture medium or cell culture medium, known in the art for contacting cultured cells. As apparent to one skilled in the art, a physiologically acceptable solution may include, but is not limited to, a phosphate buffered salt solution (PBS), a balanced salt solution (e.g., Earle's or Hank's balanced salt solution, a balanced salt solution fortified with various nutrients, and the like.

The term “cultured cells” is used herein, for the purposes of the specification and claims, to mean one or more of: cells that are cultured as anchorage-dependent or as anchorage-independent; cells comprising cellular aggregates; an organized structure or network of cells in forming a tissue, as apparent to those skilled in the art. Cells cultured as either anchorage-dependent or anchorage-independent are known to those skilled in the art to include, but are not limited to, cell lines, tumor cells, hematopoietic cells, cells isolated from a tissue, or other cell type desired to be cultured (e.g., as readily available to, or can be isolated using standard techniques by, one skilled in the art). Cellular aggregates may be comprised of a single cell type or of multiple cell types; and, in culture, may further mimic one or more functions of a tissue or organ. As apparent to one skilled in the art from descriptions herein, tissue fragments may be introduced into the cell culture device of the in vitro system according to the present invention, and the tissue fragments themselves represent a tissue, or are cultured to form a tissue using methods known in the art. Alternately, a tissue may be engineered in the cell culture device by introduction into the cell culture device of the various cell types needed to form the tissue, using standard techniques known in the art (e.g., culturing cells on a three dimensional synthetic (e.g., polyglycolic acid) or natural (e.g., collagen or extracellular) matrix). A “cell type” is used herein, for the purposes of the specification and claims, to mean cells from a given source, (e.g., a tissue or organ), or a cell in a given state of differentiation, or a cell associated with a given pathology, morphology, genetic makeup, or phenotypic expression (e.g., as determined by expression of cell determinants in forming an expression profile), as apparent to those skilled in the art. While the present invention can be used in conjunction with a wide variety of cells that can be cultured in vitro, preferred cells which may be cultured in the in vitro system according to the present invention comprise one or more cell types including, but not limited to, animal cells, insect cells, mammalian cells, human cells, transgenic cells, genetically engineered cells, transformed cells, cell lines, plant cells, anchorage-dependent cells, anchorage-independent cells, and other eukaryotic cells.

The term “secreted product” is used herein, for the purposes of the specification and claims, to mean one or more molecules released (e.g., secreted) from cultured cells. The nature of the one or molecules released will obviously depend on the cell type being cultured, and the conditions under which the cell type will produce the secreted product. As apparent to those skilled in the art, molecules which may be secreted from cultured cells include, but are not limited to, antibodies (particularly monoclonal antibodies), growth factors, enzymes, hormones, cytokines, peptides, biopharmaceuticals, nucleic acid molecules, recombinant proteins, gene products, polypeptides, metabolites, and cell-byproducts.

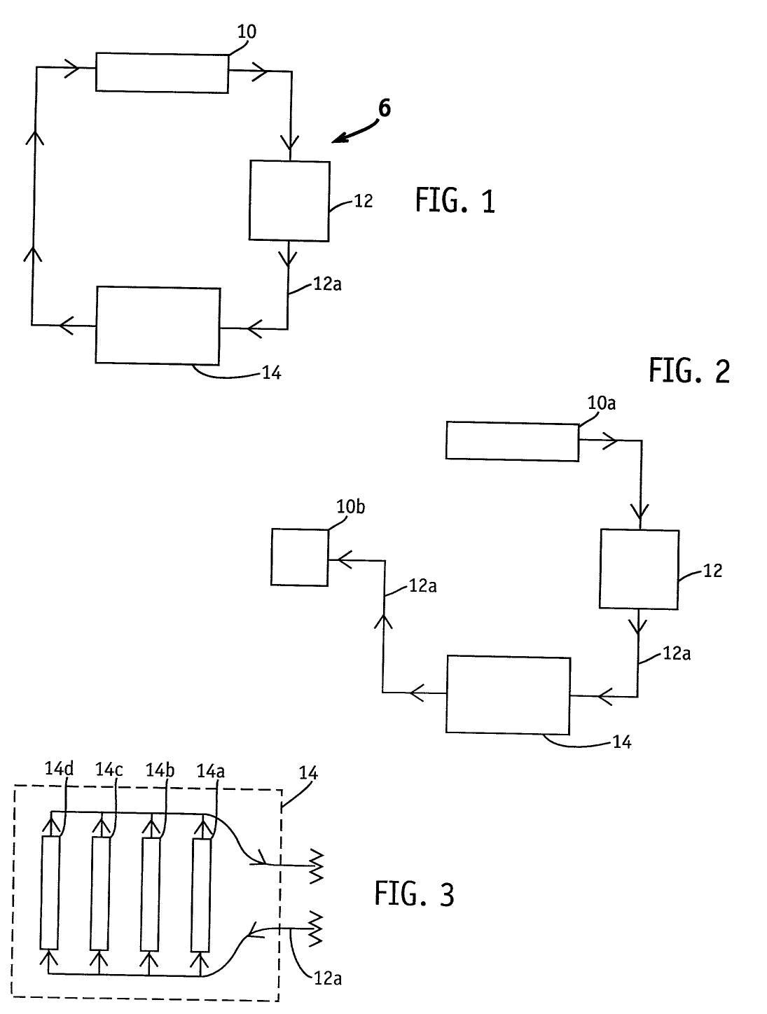

In a basic form, the in vitro system for perfusion of cultured cells according to the present invention comprises: one or more cell culture devices containing cultured cells in a medium; one or more reservoirs; and a perfusion mechanism for providing a fluid flow communication between the one or more cell culture devices and the one or more reservoirs, wherein medium is circulated in the fluid flow communication. FIG. 1 is a block diagram showing an embodiment of perfusion achievable with the in vitro system for perfusion of cultured cells according to the present invention, wherein the embodiment is known generally as a “closed loop”. In this embodiment, in vitro system 6 comprises a reservoir 10 in fluid communication with perfusion mechanism 12 and with one or more cell culture devices 14; wherein medium is circulated by the action of perfusion mechanism 12 so that the medium flows from reservoir 10 through one or more cell culture devices 14 and back to reservoir 10 (and can be recirculated through the same path of fluid communication for a desired number of cycles or desired period of time). A closed loop of medium circulation may be particularly desirable for applications which include, but are not limited to: exposing the cultured cells to a biological substance over a controlled period of time and/or with respect to a changing concentration (e.g., a decreasing concentration gradient when all or a portion of the biological substance is either consumed, metabolized, or degraded upon contact with the cultured cells); and evaluating the response of cultured cells to a biological substance or a secreted product over a controlled period of time and/or with respect to a given flow rate of medium.

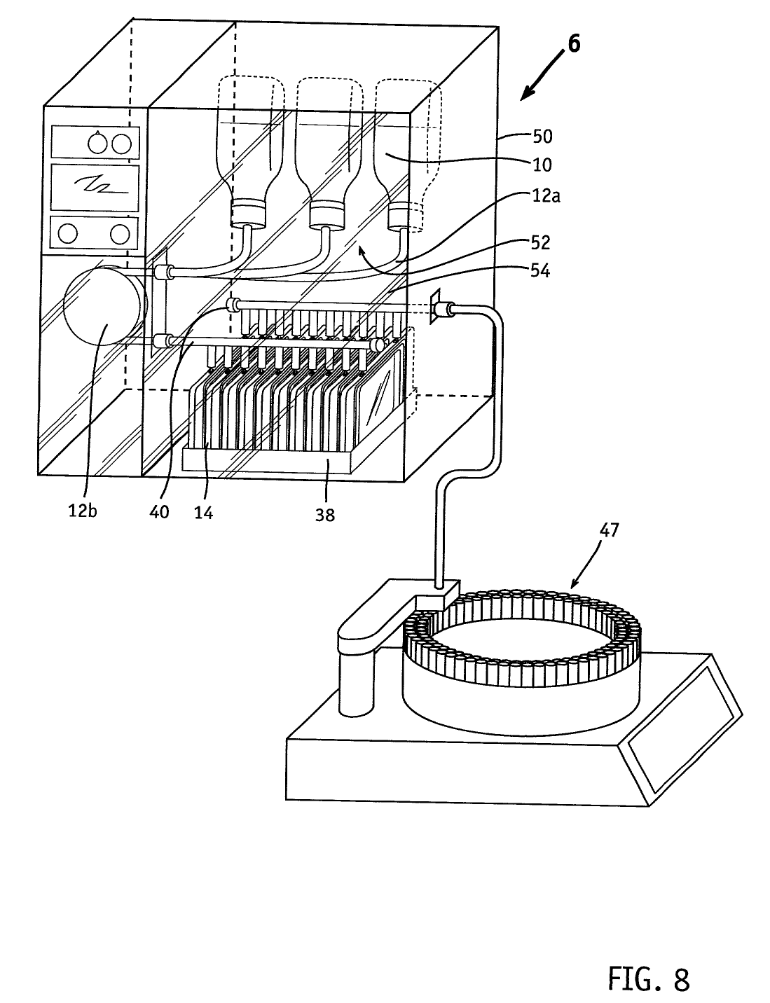

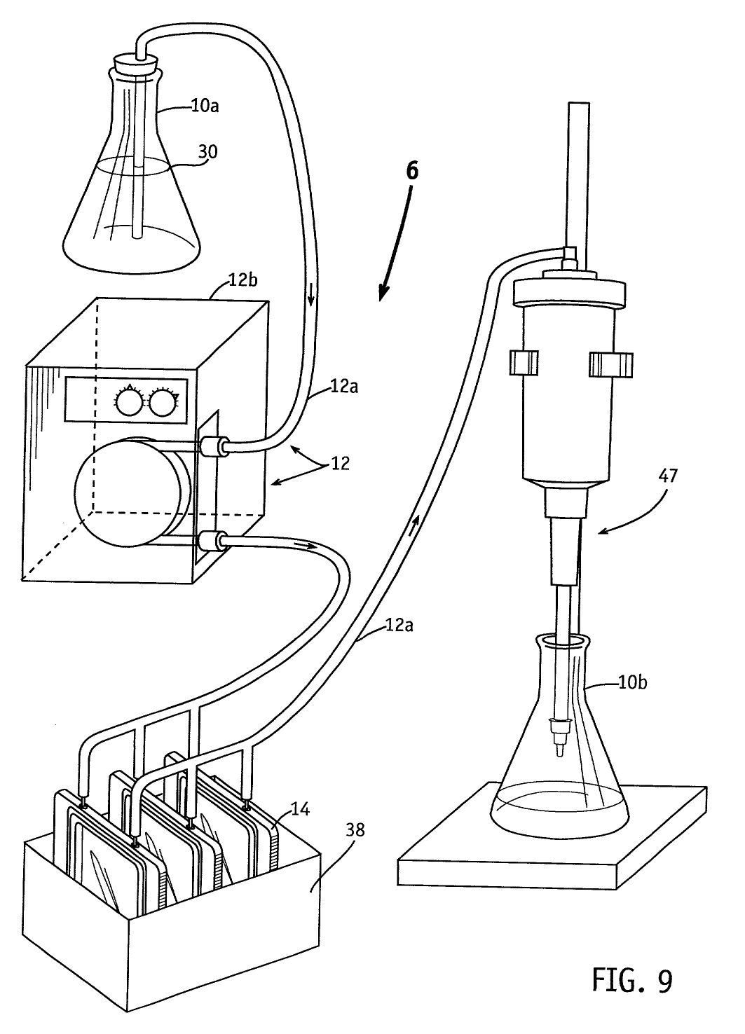

FIG. 2 is a block diagram showing an embodiment of perfusion achievable with the in vitro system for perfusion of cultured cells according to the present invention, wherein the embodiment is known generally as an “open flow”. In this embodiment, in vitro system 6 comprises a reservoir 10a in fluid communication with perfusion mechanism 12 and with one or more cell culture devices 14; wherein medium is circulated by the action of perfusion mechanism 12 so that the medium flows from reservoir 10a through one or more cell culture devices 14 and then to reservoir 10b. In the open flow embodiment, the medium flowing from the one or more cell cultures is flowed into a component of the in vitro system selected from the group consisting of a collection reservoir 10b, a harvesting mechanism (as illustrated in FIG. 8), or a combination thereof (as illustrated in FIG. 9). An open flow of medium circulation may be particularly desirable for applications which include, but are not limited to: exposing the cultured cells to a biological substance over a controlled period of time and/or with respect to a constant concentration of the biological substance; evaluating the response of cultured cells to a biological substance or a secreted product over a controlled period of time; and collection of and/or harvesting of a secreted product produced by the cultured cells.

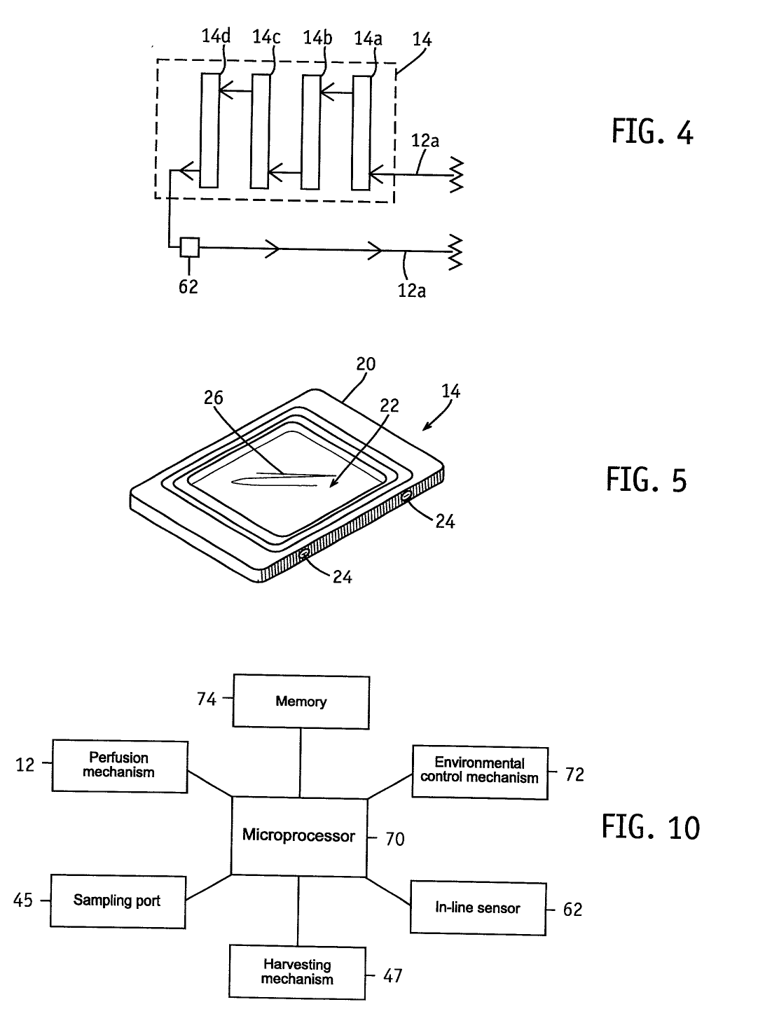

In a preferred embodiment of the in vitro system for perfusion of cultured cells illustrated in FIGS. 1 & 2, one or more cell culture devices 14 comprises a plurality of cell culture devices. FIG. 3 is an exploded view of the block diagrams illustrated in FIGS. 1 & 2, showing an embodiment of perfusion achievable using a plurality of cell culture devices 14a, 14b, 14c, and 14d in the in vitro system for perfusion of cultured cells according to the present invention, wherein the embodiment is known generally as a “parallel flow”. In a parallel flow arrangement, cultured cells in each of the plurality of the cell culture devices 14 are perfused, with medium that is flowed into and through an inlet port and which exits through an outlet port of the respective cell culture device (arrows in FIG. 3 illustrate direction of fluid flow). Thus, in an initial cycle of circulation: the medium is generally circulated through the plurality of cell culture devices at substantially the same time (i.e., there is no requirement for the medium to be first circulated through a first cell culture device before the medium can be circulated in a subsequent (relative to the fluid flow) cell culture device); and there is little or no passage of medium from one cell culture device into another cell culture device of the plurality of cell culture devices. FIG. 4 is an exploded view of the block diagrams illustrated in FIGS. 1 & 2, showing an embodiment of perfusion achievable using a plurality of cell culture devices in the in vitro system for perfusion of cultured cells according to the present invention, wherein the embodiment is known generally as a “series flow”. Note in describing this and other embodiments of the present invention, such terms as “first” and “second” and the like are words of convenience in order to distinguish between different elements. Such terms as “first” and “second” are not intended to be limiting as to the sequence of a method or priority in which the different elements may be utilized. In a series flow arrangement, a first cell culture device 14a of the plurality of the cell culture devices 14 is being perfused with medium that is flowed through its inlet port, and medium exiting through its outlet port is then flowed into a second cell culture device (e.g., 14b, via its inlet port). The medium which is flowed out of the second cell culture device (e.g., via its outlet port) may then be flowed into a third cell culture device (e.g., 14c); and this type of perfusion can continue for the desired number of cell culture devices to be in fluid communication by a series flow arrangement (i.e., it being understood that the number of cell culture devices is not critical to the invention). A particular benefit of a series flow arrangement is for evaluating the communication between different cell types, e.g., cultured in the plurality of cell culture devices is a plurality of cell types. More specifically, and for purposes of illustration but not limitation, a first cell type may be cultured in one or more cell culture devices of the plurality of cell culture devices, and a second cell type may be cultured in one or more cell culture devices (other than the one or more cell culture devices in which the first cell type is cultured) of the plurality of cell culture devices, wherein medium that has already perfused the cultured cells of the first cell type is used to contact (e.g., perfuse) cultured cells of the second cell type. In continuing with this illustrative example, if the cultured cells of the first cell type produce a secreted product that contacts and induces a response in cultured cells of the second cell type, the cultured cells of the second cell type may be evaluated for the response. If the response comprises a change in a cell parameter (e.g., including, but not limited to, growth rate, size, shape, apoptosis, differentiation, granularity, migration, light scatter, and the like), the cultured cells of the second cell type may be evaluated for that response using standard techniques known in the art for detecting and/or measuring the response. If the response comprises production of a secreted product by cultured cells of the second cell type, the medium that is flowed out from the cell culture device containing the cultured cells of the second cell type may be evaluated for that secreted product using standard techniques known in the art for detecting and/or measuring that secreted product (and, if desired, the secreted product may be further directed to a harvesting mechanism where the secreted product is purified). If the response comprises a change (physical or compositional) in the medium, the medium may be evaluated for the response (e.g., using an in-line sensor 62, as will be described herein in more detail). Alternatively, a medium containing a biological substance may be circulated through a plurality of cell culture devices, wherein some of the plurality each contain a respective cell type characteristic of a healthy tissue (e.g., kidney, liver, or lung) while the remaining of the plurality contains a cell type representative of a diseased tissue (e.g., cancer cells). In this arrangement, the effect of the biological substance on the diseased tissue and healthy tissue may be evaluated, as well as the effects of healthy tissue and/or the diseased tissue on the biological substance. It will be apparent to one skilled in the art that based on the description and illustrations herein, there are a number of ways (e.g., using a fluid flow in different priorities of the cell culture devices utilized), and applications (e.g., to evaluate one or more of adsorption, distribution, metabolism, and elimination of a biological substance in a physiologically-based model involving sequential contact with multiple cell types) for a series flow arrangement to be achieved, and these variations are intended to be encompassed by scope herein.

Referring to FIG. 5, cell culture device 14, utilized in the in vitro system for perfusion of cultured cells according to the present invention, comprises: a frame 20; a chamber 22 in which cells may be cultured; a plurality of access ports 24 (at least one access port which can comprise an inlet port, and at least one access port which can comprise an outlet port); and at least one liquid impermeable, gas permeable membrane 26, preferably forming one or more walls of chamber 22. The cell culture device is described in more detail in co-pending application Ser. Nos. 09/526006, 09/724153, and 09/724251 (the disclosures of which are herein incorporated by reference). Briefly, the cell culture device is comprised of a frame comprising a housing to which is contacted and secured taut thereto, in a leak-proof sealing arrangement, at least one gas permeable, liquid impermeable membrane (e.g., comprised of a suitable polymer). In a preferred embodiment, two liquid impermeable membranes are secured to the frame, wherein at least one of the membranes is gas permeable; and more preferably, both membranes are gas permeable. Alternatively, there is one gas permeable, liquid impermeable membrane secured to the frame with the opposing surface comprising a rigid, clear plastic material typical of conventional cell culture containers (e.g., tissue culture flask and petri dish). The gas permeable membrane is capable of allowing transfer of gases into and out of the culture chamber, and preferably is optically transparent and clear for permitting observation of the cell culture. The at least one gas permeable membrane may be secured to the frame in a leak-proof sealing using a mechanical means (e.g., heat bonding, sonic welding, pressure fit sealing, or a molding process), or a chemical means (an adhesive). As part of the cell culture device, the at least one gas permeable membrane provides an unexpected combination of properties including: efficient gas exchange, gas equilibrium, and oxygenation of cultured cells; optical transparency and clarity for observing cell culture and cell characteristics during culture; an attachment surface with gas exchange properties which promote even distribution of anchorage-dependent cells; spatial efficiency (e.g., obviates requirement for header space); and provides conditions which can promote a high rate of cell growth in achieving a high cell density in a relatively short period of time as compared to conventional cell culture devices.

The frame may be of a basic biocompatible composition that may comprise suitable plastic, thermoplastic, synthetic, or natural materials which can be fabricated into a framework structure, thereby achieving the required structural integrity for its intended purpose. In a preferred embodiment, the frame of the cell culture device further comprises an identification code comprising an identifier placed on or made a part of a frame, and which may include, but is not limited to, a bar code, a number, a series of numbers, a color, a series of colors, a letter, a series of letters, a symbol, a series of symbols, and a combination thereof. The identification code may be used for tracking and/or identifying purposes, such as to identify (e.g., for record keeping purposes) the content of cultured cells therein, or to distinguish a particular cell culture device from other cell culture device(s) in the in vitro system according to the present invention.

The culture chamber of the cell culture device, such as formed by the frame and two membranes, is accessed by at least two access ports 24 which extend between (in forming a passageway between) the outer surface of the frame and the chamber. Of the at least two access ports 24, there is an inlet port and an outlet port, each of which is self-sealing (resealable). The inlet port serves as a means by which substances (e.g., medium and/or a biological substance) may be flowed into the chamber of the cell culture device; and the outlet port serves as a means by which substances may be flowed out of the chamber of the cell culture device. In a preferred embodiment, the at least two access ports comprise a pair of access ports appearing on the same side of the cell culture device, with each access port being sealed by a septum which comprises an elastomeric material that fills all or a substantial portion of the access port, and which is sufficiently pliable to be self-sealing; e.g., thereby allowing for penetration by a tip, forming a leak-proof seal around an inserted tip, and resealing to a leak proof seal after tip withdrawal. The elastomeric material may further comprise an antimicrobial agent (e.g., triclosan or 5-chloro-2-(2,4 -dichlorophenoxy)phenol) incorporated therein to form a surface coating on the septum. An access port, particularly an access port serving as an outlet port, may further comprise a filter comprised of a biocompatible material, and of a pore size which allows for fluid flow out of the access port but which prevents cultured cells (e.g., anchorage-independent cells) from passing through the filter, in retaining the cultured cells in the chamber of the cell culture device.

In a preferred embodiment, the cell culture device is generally rectangular, and generally flat; e.g., similar to the form of a cassette. In a more preferred embodiment, the cell culture device has a length in a range of from about 10 cm to about 13.5 cm, a width in a range of from about 7 cm to about 9 cm, and a height in a range of from about 0.2 cm to about 1.0 cm. In a more preferred embodiment, the cell culture device has a length of about 12.7 cm, a width of about 8.5 cm, and a height of about 0.58 cm. Although there is no general relative restriction on either the shape or size of the culture chamber, in a preferred embodiment for culturing to achieve a high density of cells, the average distance between the two membranes is in a range of from about 0.05 to about 0.4 inches, and more preferably is in the range of from about 0.07 to about 0.08 inches.

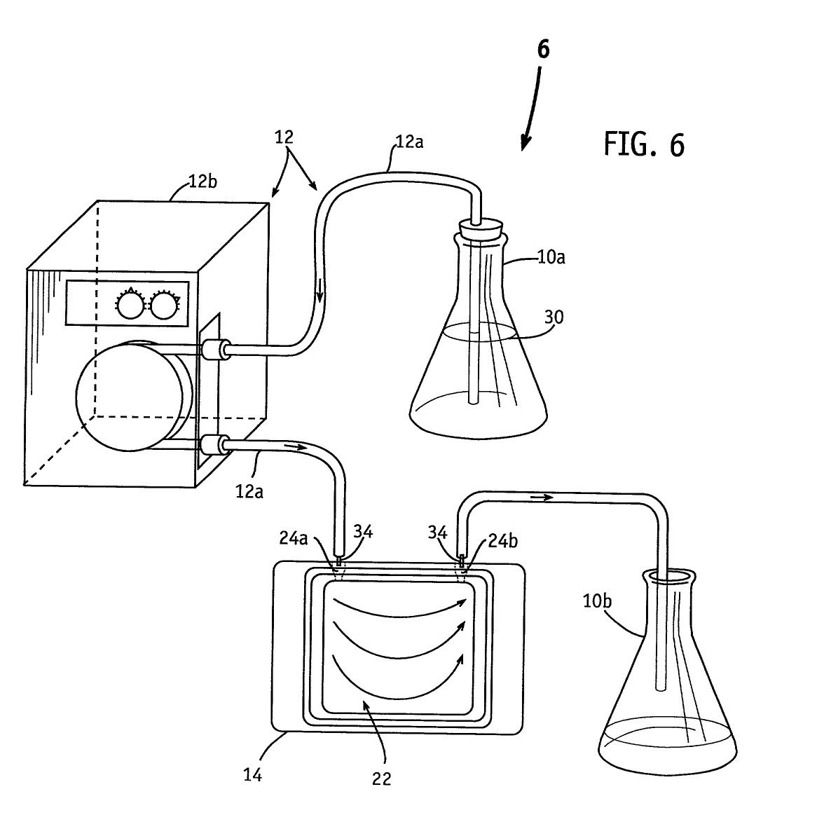

Referring to FIG. 6, the major components of the in vitro system 6 for perfusion of cultured cells according to the present invention include one or more cell culture devices 14 (preferably, containing cultured cells in a medium); one or more reservoirs 10; and a perfusion mechanism 12 for providing circulation of medium in the in vitro system, and for providing a fluid flow communication between the one or more cell culture devices and the one or more reservoirs. A reservoir 10 is any suitable containment means for containing a fluid such as a medium. For example, as apparent to one skilled in the art, a reservoir may be a container that includes, but is not limited to, a flask, a bottle, a bag adapted for holding fluids (e.g. intravenous fluid bag), a flask, a tube, a vial, and the like. In a preferred embodiment, the reservoir is closed or sealable so as to prevent microbial contamination of medium comprising a sterile medium contained therein. In continuing with reference to FIG. 6, illustrated is an open flow embodiment of the in vitro system 6 according to the present invention. In this open flow embodiment, reservoir 10a, containing sterile medium 30, is in fluid communication with perfusion mechanism 12 and with one or more cell culture devices 14. In a preferred embodiment, perfusion mechanism 12 comprises a pump 12b, and tubing 12a operatively connected to pump 12b. As apparent to one skilled in the art, a variety of pumps may be used in conjunction with the in vitro system according to the present invention. A suitable pump may include, but is not limited to, a peristaltic pump, a roller pump, and the like. Preferably, the pump may further comprise controls for controlling the direction and velocity of the medium being pumped. Tubing 12a allows for the flow of a fluid (e.g., medium) therethrough in providing fluid communication between one or more reservoirs 10 and one or more cell culture devices 14. Preferably, the tubing is comprised of a material that is sterilizable, and more preferably is comprised of a flexible polymer. As the specific character of the material which comprises the tubing does not, in and of itself, constitute the subject matter of the instant invention, it should be apparent to one skilled in the art that a wide latitude of choice can be exercised in selecting suitable material having properties compatible with its intended purpose. In operation, pump 12b causes medium 30 to be pumped from reservoir 10a into, and along the fluid pathway provided by, tubing 12a. Thus, medium 30 is circulated by the action of perfusion mechanism 12 so that medium 30 flows from reservoir 10a through one or more cell culture devices 14 and then to reservoir 10b (arrows in FIG. 6 are illustrative of direction of fluid flow). As illustrated in FIG. 6, at least two access ports 24 are provided to form passageways between the outer surface of the frame and the chamber of cell culture device 14. Preferably, the access ports 24 each comprise a resealable septum for receiving a rigid end or tip 34 operatively connected to tubing 12a, and for forming a leak-proof seal around an inserted tip 34. Medium 30 is pumped through tubing 12a, flowed through inlet port 24a and into chamber 22 of cell culture device 14. The flow rate of medium, as pumped by perfusion mechanism 12, is of a sufficient flow rate to flow medium 30 through inlet port 24a, into chamber 22 where the medium is circulated, and out through outlet port 24b. Thus, for example, where cell culture device contains cultured cells and cell culture medium, medium 30 is flowed into the chamber and contacts the cultured cells in mixing with cell culture medium. The resultant mixture of medium is flowed out through the outlet port, in and along the fluid path provided by tubing 12a, and into collection reservoir 10b for collecting the medium. While cell culture device is shown as being in a vertical position (“on edge”) for illustrative purposes in FIG. 6, it is apparent to one skilled in the art that in this and any other embodiments described herein, the cell culture device may be positioned in any one of several positions (e.g., laying flat in a horizontal position on a surface). As will be described in more detail herein, this and other embodiments of the in vitro system according to the present invention may further comprise a housing for enclosing the in vitro system, and which may further provide a controlled environment.

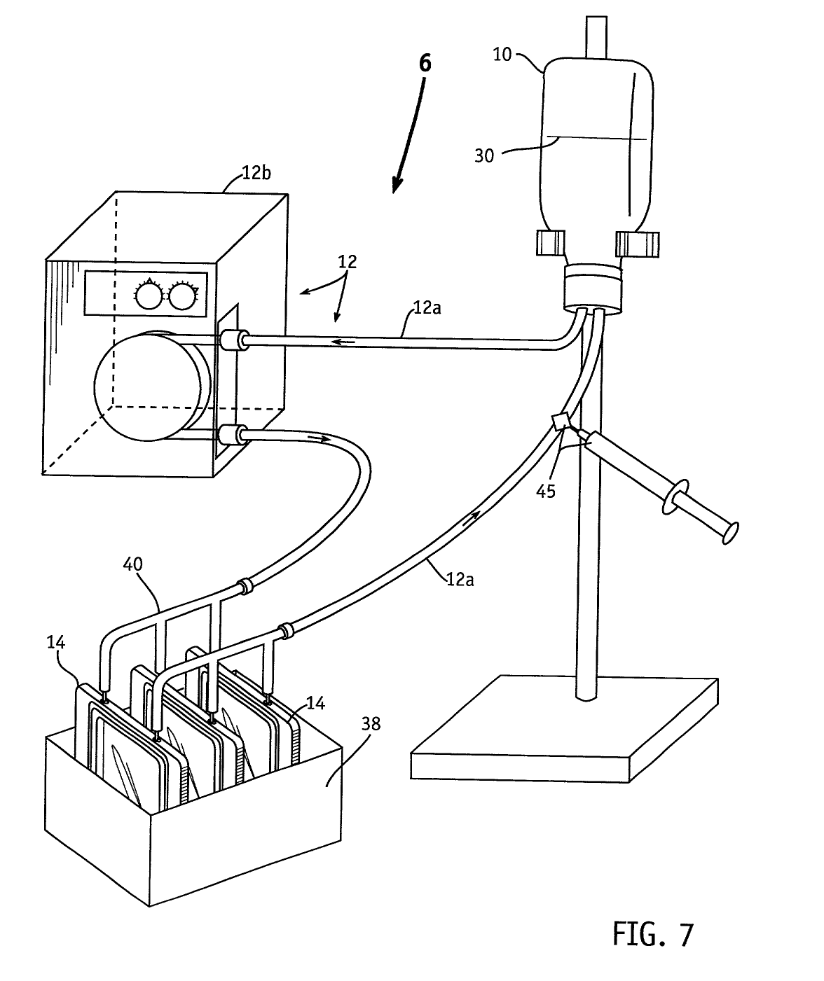

Referring to FIG. 7, the major components of the in vitro system 6 for perfusion of cultured cells according to the present invention include one or more cell culture devices 14 (preferably, containing cultured cells in a medium); one or more reservoirs 10; and a perfusion mechanism 12. The in vitro system according to the present invention may further comprise one or more additional components. For example, the in vitro system may further comprise a component selected from the group consisting of a rack for accommodating the one or more cell culture devices, one or more manifolds for regulating fluid flow, a sampling port by which a sample may be withdrawn from the fluid flow, a harvesting mechanism in operative communication with the fluid flow (e.g., for harvesting a secreted product from the medium flowed out of the one or more cell culture devices), one or more in-line sensors in operative communication with the fluid flow, a housing, a microprocessor, and a combination thereof. In continuing with reference to FIG. 7, illustrated is a closed flow embodiment of the in vitro system 6 according to the present invention. In this closed flow embodiment, reservoir 10, containing sterile medium 30, is in fluid communication with perfusion mechanism 12 and with one or more cell culture devices 14. In this illustration, one or more cell culture devices 14 comprises a plurality of cell culture devices. It will be apparent to one skilled in the art that a rack is preferably used to accommodate one or more cell culture devices, and particularly preferable when a plurality of cell culture devices is used in the system. As illustrated in FIG. 7, rack 38 comprises a housing having an open side in forming a chamber into which can be inserted the one or more cell culture devices. Preferably the rack further comprises ledges along which a cell culture device can be guided and snugly received, and also securedly held into position during use. A preferred rack for accommodating one or more cell culture devices is described in more detail in copending application Ser. No. 09/697920 (the disclosure of which is herein incorporated by reference). As apparent to one skilled in the art, a rack 38 may comprise a suitable rigid material, having the required structural integrity for its intended purpose, which can be fabricated to accommodate a plurality of cell culture devices 14. It will also be apparent to one skilled in the art that a variety of materials and designs may be suitable in fabricating a rack for use with the in vitro system according to the present invention.

As illustrated in FIG. 7, perfusion mechanism 12 (comprising pump 12b, and tubing 12a) causes medium 30 to be pumped from reservoir 10 into, and along the fluid pathway provided by, perfusion mechanism 12. Thus, medium 30 is circulated by the action of perfusion mechanism 12 so that medium 30 flows from reservoir 10, through a plurality of cell culture devices 14, and then back and into reservoir 10 (arrows in FIG. 7 are illustrative of direction of fluid flow). As illustrated in FIG. 7, the perfusion mechanism 12 may further comprise one or more manifolds 40 to regulate the flow of the medium, particularly regulating the flow with respect to a cell culture device 14. A manifold particularly suitable for use with a plurality of cell culture devices comprises a number of orifices, each of which is aligned to be in fluid communication with an access port of a cell culture device. Fluid flow may be regulated, for example, by the size of a manifold orifice, the length of the fluid communication between the manifold and an access port of the cell culture device, or other means apparent to one skilled in the art. Manifold 40 may further comprise controllable valves optionally disposed in the manifold to further regulate fluid flow. For example, the valve may selectively and adjustably reduce the size of the fluid flow communication between the manifold and a cell culture device in regulating the fluid flow to that cell culture device. Thus, preferably the flow of medium into and/or out of each individual cell culture device in the in vitro system according to the present invention may be separately controlled (e.g., regulated with respect to one or more of speed, pressure, and flow). This preferred embodiment may be particularly utilized when each of the plurality of cell culture devices contains cultured cells representative of a cell type or tissue, and the fluid flow rates in each cell culture device and/or between the plurality of cell culture devices is biologically based to model the flow rates between and among a corresponding biological organ, tissue, etc. In a preferred embodiment, there is a plurality of manifolds: a first manifold to regulate the flow of medium from reservoir 10 to cell culture devices 14, and a second manifold to regulate the flow of medium returning to reservoir 10 from cell culture devices 14. Thus, there is a manifold for regulating the flow rate of medium into an inlet port of a cell culture device or into inlet ports of respective cell culture devices of a plurality of cell culture devices in contacting cultured cells; and a manifold for regulating the flow rate of medium out of an outlet port of a cell culture device or out of outlet ports of respective cell culture devices of a plurality of cell culture devices.

The in vitro system for perfusion of cultured cells according to the present invention may further comprise a sampling port. A sampling port refers to any device for obtaining a sample of medium from the fluid pathway of the system according to the present invention. More preferably, a sampling port is provided for the removal of an aliquot of medium at a desired point in the fluid flow of the system. For example, the sampling port may include, but is not limited to, a valve for diverting an aliquot of the medium, a shunt for diverting an aliquot of medium, a syringe, and a combination thereof. Further, the sampling port may be provided with a variety of couplings for connecting with various fittings to achieve its intended purpose. As illustrated in FIG. 7, sampling port 45 (comprising a valve and syringe combination) is positioned to remove an aliquot of medium prior to its flow into reservoir 10. Sampling at this point in the fluid flow, or any other desired point in the fluid flow, of the system allows a user of the system to determine the concentration of an analyte (e.g., specific nutrient of the medium, or a biological substance, or a secreted product, or a combination thereof) at the desired point in the fluid pathway. In a preferred embodiment, a sampling port is positioned at a point in the fluid flow between two components of the system according to the present invention that are in fluid communication with respect to each other; and the point may include, but is not limited to, between two cell culture devices in a plurality of cell culture devices, between a reservoir and a cell culture device (e.g., prior to the flow of the medium into the cell culture device), between a cell culture device and a reservoir (e.g., after the medium is flowed out of a cell culture device but before it is flowed into the reservoir), and a combination thereof (e.g., multiple sampling ports, each allowing sampling at a different point in the fluid flow of the system).

The in vitro system for perfusion of cultured cells according to the present invention may further comprise a harvesting mechanism for harvesting a secreted product from the fluid flow of the system. A harvesting mechanism may include, but is not limited to, a fraction collector, a chromatography column, a combination thereof, and any other device known in the art for harvesting a secreted product. For example, the harvesting mechanism may comprise a chromatography system known in the art (e.g., HPLC-high pressure liquid chromatography, FPLC-fast pressure liquid chromatography, and the like) which is in fluid communication with the fluid flow output (comprising medium which is flowed from the one or more cell culture device; i.e., after having been in contact with the cultured cells) of the in vitro system according to the present invention. As illustrated in FIG. 8, in one embodiment harvesting mechanism 47 comprises a fraction collector, a device utilized for collecting liquid samples originating from a fluid flow. A commercially available fraction collector may be utilized to collect fractions of the fluid flow output coming from (e.g., after having flowed through) the one or more cell culture devices. Generally, a fraction collector collects fractions of the fluid flow output (e.g., medium containing a secreted product) in individual collection tubes for a given time interval or a certain predetermined number of droplets of the fluid flow output. Thus, discrete fractions of the fluid flow output may be collected in separate collection containers for later analysis or use.

In this and other embodiments of the present invention, and as illustrated in FIG. 8, the in vi tro system for perfusion of cultured cells may further comprise a housing 50. Housing 50 forms an enclosure or chamber 52 which can contain one or more components of the in vitro system 6 according to the present invention. The housing 50 may comprise walls, and at least one access 54 comprising a securable, sealable panel or door which can be opened to access chamber 52 or closed to form a closed environment for chamber 52. The panel may comprise a transparent material (glass; or a clear synthetic resin, e.g., plexiglass) which allows viewing of the contents of the chamber without breaching the chamber environment. The housing may further include appropriate electronics (e.g., microprocessor, memory, display, and other circuit components) suitable for their intended purpose as apparent to those skilled in the art. The housing may further comprise an environmental control mechanism that may control the environment for the cultured cells by controlling one or more of temperature, atmospheric gas content (e.g., CO₂, O₂), humidity (water vapor content), pressure, sterility, and the like, in the chamber. Preferably, the environment control mechanism includes, but is not limited to, a heating mechanism, a humidity control mechanism, a CO₂ controller (e.g., CO₂ tank, valve, and sensor); and may further comprise a controlling pressure/airflow mechanism preferably including a pressure pump means or blower means (e.g., preferably, for providing a laminar flow of filtered air); such as by using standard components of typical tissue culture incubators as known to those skilled in the art of cell culture. As apparent to one skilled in the art, desired environmental conditions for culturing cells include a desired temperature in the range of about 35° C. to about 42° C., and more preferably about 37° C.; and may further comprise a CO₂ content of about 5%. In normal operation, and as illustrated in FIG. 10, environmental control mechanism 72 may be controlled by microprocessor 70 for programming operations in providing a controlled environment for the cultured cells housed in chamber 52 of in vitro system 6 according to the present invention.

As illustrated in FIG. 9, in another embodiment harvesting mechanism 47 comprises a chromatography column in providing liquid chromatography for separation (harvesting) of a secreted product in or from the fluid flow of the in vitro system according to the present invention. In one illustration of this embodiment, the chromatography column allows the secreted product (desired to be separated) to flow through the column while being separated by size or chemical property from other components in the fluid flow (e.g., components of the medium other than the secreted product). A fraction collector may be used to collect fractions of fluid passing through the column. In another illustration of this embodiment, a chromatography column contains a separation medium that retains a secreted product desired to be separated. For example, the column separation material may selectively bind (known in the art as an “affinity matrix”, or an “ionic matrix”) or trap (e.g., known in the art as “size exclusion matrix”) the secreted product desired to be separated. In this illustration, the fluid flow output (e.g., medium coming from, after having flowed through, the one or more cell culture devices, and containing the secreted product) is flowed into a chromatography column, wherein the secreted products desired to be separated is retained in the chromatography column, while the remainder of the fluid flow is passed through the column as effluent. Secreted product, retained in the column, may then be eluted by flowing an elution solution (a solution known in the art as functioning to elute the secreted product from the column matrix) through the column. The eluted secreted product may then be collected in one or more containers for storage or further analysis. Alternatively, a fraction collector may be used to collect fractions of the eluate from the chromatographic column. In a preferred embodiment of harvesting a secreted product using the in vitro system according to the present invention, the one or more cell culture devices contain cultured cells comprising a hybridoma cell line producing a secreted product comprising a monoclonal antibody. The monoclonal antibody is harvested from the medium by use of an affinity column (e.g., protein A column) followed by elution from the column using an appropriate elution solution as known in the art.

As illustrated in FIG. 4, the in vitro system according to the present invention may further comprise one or more sensors 62 which can be any conventional liquid sensing device known in the art. For example, at a desired point in the fluid flow of the system, characteristics of the fluid flow can be evaluated. A flow meter can measure flow rate, and thus allow precise control over the flow rate(s) within the system. One or more in-line sensors may be in operative communication with the fluid flow in allowing measurement of a physical or compositional characteristic of the medium flowing through the evaluation point. Examples of such devices include, but are not limited to, a pH sensor, a CO₂ sensor, a turbidity sensor, a flow photometer which measures the optical density of the medium at suitable wavelengths (e.g., typically in a range of from about 254 nm to about 280 nm), and a combination thereof. For example, detecting a light absorbance relating directly to the presence and/or concentration of a particular species of secreted product in the medium may be used to distinguish between the different species of secreted products that may be present in the medium, as well as to determine under what conditions a specific secreted product is produced during the process of using the in vitro system according to the present invention. Similarly, a response of cultured cells to a biological substance may be a response which alters the pH of the medium flowing out of the one or more cell culture devices containing cultured cells treated with a biological substance. Thus, an in-line pH sensor, which is in operative communication with the medium flowing out of the one or more cell culture devices, may be used for detecting a change in pH. In that regard, a corresponding in-line pH sensor may be placed in operative communication with the fluid flow before the biological substance contacts the cultured cells in generating a baseline pH value to be compared with the value obtained by an in-line sensor which is in operative communication with the medium flowing out of the one or more cell culture devices.

The in vitro system according to the present invention may further comprise a microprocessor 70. In referring to FIG. 10, microprocessor controls and coordinates the operation of the in vitro system according to the present invention, and provides for data storage (e.g., in memory) related to programming, functions, and collection of data (e.g., resulting from environmental control mechanism 72, in-line sensors 62, and the like). Preferably, programmable commands from the user are inputted into the microprocessor 70 via a keyboard and/or any additional control buttons (including a touch-sensitive display). Information regarding the operation, or programming, or function, or a combination thereof, of the in vitro system according to the present invention (e.g., relative to one or more of: environmental control mechanism 72, or in-line sensor 62, or perfusion mechanism 12, or sampling port 45, or harvesting mechanism 47) may be displayed on a display panel, and may be stored in memory 74. As apparent to one skilled in the art, suitable components of microprocessors (including circuitry, data storage drive, display, and keyboard) are conventional in the art. Microprocessor 70 may be built into the in vitro system according to the present invention, or may comprise a host computer (e.g., typical workstation, or personal computer, or other suitable computer platform) in operative communication with the in vitro system according to the present invention.

In a method of using the in vitro system for perfusion of cultured cells according to the present invention, a biological substance may be contacted with the cultured cells in the in vitro system by: (a) mixing the biological substance with a medium; and (b) circulating the medium containing the biological substance through the one or more cell culture devices of the in vitro system, wherein the biological substance contacts cultured cells contained in the one or more cell culture devices. For example, preferably each of the one or more cell culture devices 14 contains cultured cells in a culture medium in chamber 22 of the cell culture device. The medium in reservoir 10 contains a biological substance to be tested. The medium is circulated by the action of perfusion mechanism 12, and through the fluid pathway provided by perfusion mechanism 12, into the one or more cell culture devices 14. More particularly, the medium is circulated into and then out of (“through”) the one or more cell culture devices in bringing the biological substance in contact with the cultured cells. The medium, upon exiting the one or more cell culture devices may then be flowed into a component selected from the group consisting of a reservoir, a sampling port, a harvesting mechanism, and a combination thereof. In one embodiment, the one or more cell culture devices comprises a plurality of cell culture devices. The plurality of cell culture devices may contain cultured cells of the same cell type. Alternatively, the plurality of cell culture devices contains cultured cells of a different cell type; e.g., each cell culture device of the plurality of cell culture devices contains a cell type that is not contained in the other cell culture devices of the plurality of cell culture devices. The method may further comprise measuring a response of the cultured cells to the biological substance (e.g., as a result of exposure to the biological substance). The response of the cultured cells may be detected by evaluating a cell parameter, a parameter in the medium, or a combination thereof. A cell parameter (e.g., including, but not limited to, one or more of: growth rate, size, shape, apoptosis, differentiation, granularity, migration, light scatter, and the like) may be evaluated using standard methods known in the art. Evaluating the cell parameter may be achieved by measuring the cell parameter before the cultured cells are exposed to the biological substance (the measurement resulting in a “baseline value”), measuring the same cell parameter after the cultured cells have been exposed to the biological substance (the measurement resulting in a “test value”), and comparing the baseline value with the test value, wherein a difference between the baseline value and the test value may be indicative of a response of the cultured cells to the biological substance. A parameter in the medium may include one or more of: the presence of a secreted product released by cultured cells as a result of exposure to the biological substance (e.g., the biological substance contacts the cultured cells and induces the production of the secreted product), the presence of one or more metabolites of the biological substance, concentration of one or more ions (e.g., calcium, magnesium, and the like), concentration of one or more gases (e.g., oxygen, carbon dioxide, and the like), pH, concentration of one or more nutrients (e.g., glucose), and the like. A parameter of the medium may be evaluated using standard methods known in the art for measuring the parameter. Evaluating the parameter of the medium may be achieved by measuring the parameter of the medium before the medium (and the biological substance) is contacted with the cultured cells (the measurement resulting in a “baseline value”), measuring the same parameter in medium which has been in contact with the cultured cells (e.g., from the fluid flow after having passed through the one or more cell culture devices, and also described herein as the fluid flow output) (the measurement resulting in a “test value”), and comparing the baseline value with the test value, wherein a difference between the baseline value and the test value may be indicative of a response of the cultured cells to the biological substance.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

What is claimed:

1. An in vitro system for perfusion of cultured cells comprising:
(a) one or more cell culture devices, wherein each cell culture device of the one or more cell culture devices comprises a frame, a chamber for culturing cells, a plurality of access ports, and at least one gas permeable, liquid impermeable membrane;
(b) one or more reservoirs; and
(c) a perfusion mechanism for providing a fluid flow between the one or more cell culture devices and the one or more reservoirs.

2. The in vitro system according to claim 1, wherein a reservoir of the one or more reservoirs contains a medium which is flowed from the reservoir and through the one or more cell culture devices, and wherein the chamber of each of the one or more cell culture devices comprises cultured cells.

3. The in vitro system according to claim 2, wherein the chamber of each of the one or more cell culture devices further comprises a medium.

4. The in vitro system according to claim 2, wherein the medium is circulated through the one or more cell culture devices by:
flowing the medium through an access port comprising an inlet port of, and into the chamber of, each of the one or more cell culture devices so that medium contacts the cultured cells; and
flowing medium out of the chamber of, and through an access port comprising an outlet port of, each of the one or more cell culture devices.

5. The in vitro system according to claim 4, wherein the medium comprises a biological substance and a fluid selected from the group consisting of tissue culture medium, cell culture medium, a physiologically acceptable solution, and a combination thereof.

6. The in vitro system according to claim 2, wherein the one or more cell culture devices comprises a single cell culture device.

7. The in vitro system according to claim 6, wherein the cultured cells comprise a single cell type.

8. The in vitro system according to claim 6, wherein the cultured cells comprise a plurality of cell types.

9. The in vitro system according to claim 6, wherein the medium is circulated in the in vitro system in a closed loop; wherein the one or more reservoirs comprises a reservoir containing a medium; and wherein the closed loop comprises flowing the medium from the reservoir through the cell culture device and in contact with the cultured cells, and back into the reservoir.

10. The in vitro system according to claim 9, wherein the medium is flowed through the cell culture device by flowing the medium into an access port comprising an inlet port of, and into the chamber of, the cell culture device so that medium contacts the cultured cells; and flowing the medium out of the chamber, and through an access port comprising an outlet port, of the cell culture device.

11. The in vitro system according to claim 9, wherein the medium comprises a biological substance, and a fluid selected from the group consisting of tissue culture medium, cell culture medium, a physiologically acceptable solution, and a combination thereof.

12. The in vitro system according to claim 11, wherein the medium further comprises a secreted product produced by the cultured cells.

13. The in vitro system according to claim 6, wherein the medium is circulated in the in vitro system in an open flow; wherein the one or more reservoirs comprises a reservoir containing a medium; wherein the in vitro system further comprises a component selected from the group consisting of a collection reservoir, a harvesting mechanism, and a combination thereof; and wherein the open flow comprises flowing the medium from the reservoir containing the medium through the cell culture device and in contact with the cultured cells, and flowing the medium from the cell culture device and into a component of the in vitro system, wherein the component comprises a component selected from the group consisting of a collection reservoir, a harvesting mechanism, and a combination thereof.

14. The in vitro system according to claim 13, wherein the medium is flowed through the cell culture device by flowing the medium into an access port comprising an inlet port of, and into the chamber of, the cell culture device so that medium contacts the cultured cells; and flowing the medium out of the chamber, and through an access port comprising an outlet port, of the cell culture device.

15. The in vitro system according to claim 13, wherein the medium comprises a biological substance, and a fluid selected from the group consisting of tissue culture medium, cell culture medium, a physiologically acceptable solution, and a combination thereof.

16. The in vitro system according to claim 13, wherein the medium flowing from the cell culture device further comprises a secreted product.

17. The in vitro system according to claim 2, wherein the one or more cell culture devices comprises a plurality of culture devices.

18. The in vitro system according to claim 17, wherein perfusion of the cultured cells contained within the plurality of cell culture devices comprises a parallel flow arrangement.

19. The in vitro system according to claim 17, wherein perfusion of the cultured cells contained within the plurality of cell culture devices comprises a series flow arrangement.

20. The in vitro system according to claim 17, wherein cultured in the plurality of cell culture devices are cultured cells comprising a single cell type.

21. The in vitro system according to claim 17, wherein cultured in the plurality of cell culture devices are cultured cells comprising a plurality of cell types.

22. The in vitro system according to claim 17, wherein the medium is circulated in the in vitro system in a closed loop; wherein the one or more reservoirs comprises a reservoir containing a medium; and wherein the closed loop comprises flowing the medium from the reservoir through the plurality of cell culture devices in contacting the cultured cells, and back into the reservoir.

23. The in vitro system according to claim 22, wherein the medium is flowed through the plurality of cell culture devices by flowing the medium into an access port comprising an inlet port of, and into the chamber of, each of the plurality of cell culture devices so that medium contacts the cultured cells; and flowing the medium out of the chamber, and through an access port comprising an outlet port, of each of the plurality of cell culture devices.

24. The in vitro system according to claim 22, wherein the medium comprises a biological substance, and a fluid selected from the group consisting of tissue culture medium, cell culture medium, a physiologically acceptable solution, and a combination thereof.

25. The in vitro system according to claim 22, wherein the medium further comprises a secreted product produced by the cultured cells.

26. The in vitro system according to claim 17, wherein the medium is circulated in the in vitro system in an open flow; wherein the one or more reservoirs comprises a reservoir containing a medium; wherein the in vitro system further comprises a component selected from the group consisting of a collection reservoir, a harvesting mechanism, and a combination thereof; and wherein the open flow comprises flowing the medium from the reservoir containing the medium through the plurality of cell culture devices in contacting the cultured cells with the medium, and flowing the medium from the plurality of cell culture devices and into a component of the in vitro system, wherein the component comprises a component selected from the group consisting of a collection reservoir, a harvesting mechanism, and a combination thereof.

27. The in vitro system according to claim 26, wherein the medium is flowed through the plurality of cell culture devices by flowing the medium into an access port comprising an inlet port of, and into the chamber of, each of the plurality of cell culture devices so that medium contacts the cultured cells; and flowing the medium out of the chamber, and through an access port comprising an outlet port, of each of the plurality of cell culture devices.

28. The in vitro system according to claim 26, wherein the medium comprises a biological substance, and a fluid selected from the group consisting of tissue culture medium, cell culture medium, a physiologically acceptable solution, and a combination thereof.

29. The in vitro system according to claim 26, wherein the medium flowing from the cell culture device further comprises a secreted product.

30. The in vitro system according to claim 1, further comprising a component selected from the group consisting of a rack for accommodating the one or more cell culture devices, one or more manifolds for regulating fluid flow, a sampling port by which a sample may be withdrawn from the fluid flow, a harvesting mechanism in operative communication with the fluid flow, one or more in-line sensors in operative communication with the fluid flow, a housing, a microprocessor, and a combination thereof.

31. The in vitro system according to claim 30, wherein the one or more manifolds comprises a plurality of manifolds comprising: a manifold for regulating the flow rate of medium into the one or more cell culture devices; and a manifold for regulating the flow rate of medium out of the one or more cell culture devices.

32. The in vitro system according to claim 30, wherein the housing further comprises an environmental control mechanism.

33. A method of using the in vitro system according to claim 2 for contacting a biological substance with cultured cells, the method comprising: (a) mixing the biological substance with the medium; and (b) circulating the medium containing the biological substance through the one or more cell culture devices, wherein the biological substance contacts cultured cells contained in the one or more cell culture devices.

34. The method according to claim 33, further comprising flowing medium which had been circulated through the one or more cell culture devices into a component selected from the group consisting of a reservoir, a sampling port, a harvesting mechanism, and a combination thereof.

35. The method according to claim 33, wherein the one or more cell culture devices comprises a plurality of cell culture devices.

36. The method according to claim 35, wherein cultured in the plurality of cell culture devices are cultured cells comprising a single cell type.

37. The method according to claim 35, wherein the cultured in the plurality of cell culture devices are cultured cells comprising a plurality of cell types.

38. The method according to claim 33, further comprising measuring a response of the cultured cells to the biological substance by evaluating a parameter selected from the group consisting of a cell parameter, a parameter in the medium, or a combination thereof; wherein the parameter is measured before the cultured cells are contacted with the biological substance in generating a baseline value; wherein the parameter is also measured after the cultured cells have been contacted with the biological substance in generating a test value; and comparing the baseline value with the test value, wherein a difference between the baseline value and the test value is indicative of a response of the cultured cells to the biological substance.
